(19) United States
(12) Patent Application Publication
(10) Pub. No.: US 2022/0291116 A1
(43) Pub. Date: Sep. 15, 2022

(54) GAS SUPPLY AND DISCHARGE ADAPTER AND GAS DETECTION DEVICE

(71) Applicant: KYOCERA Corporation, Kyoto-shi, Kyoto (JP)

(72) Inventors: Daisuke UEYAMA, Nara-shi, Nara (JP); Shinichi ABE, Uji-shi, Kyoto (JP)

(21) Appl. No.: 17/636,853

(22) PCT Filed: Aug. 28, 2020
(86) PCT No.: PCT/JP2020/032687

§ 371 Date: Feb. 18, 2022

(30) Foreign Application Priority Data
Aug. 29, 2019 (JP) 2019-157329

(51) Int. Cl. G01N 21/05; G01N 21/3518

(52) U.S. Cl. CPC G01N 21/05; G01N 21/3518; G01N 2021/3527

(57) ABSTRACT

A gas supply and discharge adapter has an opening through which a gas flows into and out of a gas sensor, an inflow port through which the gas flows into the opening, and an outflow port through which the gas flows out of the opening. The inflow port and the outflow port are positioned independently of each other. When the inflow port and the opening are viewed in plan through the gas supply and discharge adapter, the inflow port partially overlaps the opening.

### CROSS REFERENCE TO RELATED APPLICATION

This application claims the benefit of Japanese Patent Application No. 2019-157329 filed Aug. 29, 2019, which is hereby incorporated by reference herein in its entirety.

### TECHNICAL FIELD

The present disclosure relates to a gas supply and discharge adapter and a gas detection device.

### BACKGROUND ART

Sensors, such as odor sensors to detect an odor and gas concentration sensors to detect the concentration of a gas, are hitherto known. For example, when a smell of air needs to be measured, known sensors detect odorous substances contained in the air. Such sensors are hereinafter referred to as “gas sensors”.

When a gas sensor is used to detect a gas, a device is also used to cause the target gas to flow into or out of the gas sensor. Various types of such devices have been proposed. For example, PTL 1 discloses a gas supply adapter that has an inlet duct for feeding a gas into a chamber and an outlet to vent the gas from the chamber.

### CITATION LIST

### Patent Literature

PTL 1: Japanese Unexamined Patent Application Publication (Translation of PCT Application) No. 2005-537488

### SUMMARY OF INVENTION

A gas supply and discharge adapter according to one embodiment has an opening through which a gas flows into and out of a gas sensor, an inflow port through which the gas flows into the opening, and an outflow port through which the gas flows out of the opening. The inflow port and the outflow port are positioned independently of each other. When the inflow port and the opening are viewed in plan through the gas supply and discharge adapter, at least a portion of the inflow port overlaps the opening.

A gas detection device according to one embodiment has a gas sensor that detects a gas supplied to an opening, an inflow port through which the gas flows into the opening, and an outflow port through which the gas flows out of the opening. The inflow port and the outflow port are positioned independently of each other. When the inflow port and the opening are viewed in plan through the gas supply and discharge adapter, at least a portion of the inflow port overlaps the opening.

### BRIEF DESCRIPTION OF DRAWINGS

FIG. 1 is a perspective view illustrating the exterior of a gas supply and discharge adapter according to one embodiment.

FIG. 2 is a front view illustrating the exterior of a gas supply and discharge adapter according to one embodiment.

FIG. 3 is a plan view illustrating the exterior of a gas supply and discharge adapter according to one embodiment.

FIG. 4 is a bottom view illustrating the exterior of a gas supply and discharge adapter according to one embodiment.

FIG. 5 is a plan view illustrating the exterior of a gas supply and discharge adapter according to one embodiment.

FIG. 6 is a plan view illustrating the exterior of a gas supply and discharge adapter according to one embodiment.

FIG. 7 is a perspective view illustrating the exterior of a gas supply and discharge adapter according to one embodiment.

FIG. 8 is a plan view illustrating the exterior of a gas supply and discharge adapter according to one embodiment.

FIG. 9 is a front view illustrating the exterior of a gas detection device according to one embodiment.

### DESCRIPTION OF EMBODIMENTS

In the present disclosure, the “gas detection device” may be a device for detecting a gas flowing through a channel. In the present disclosure, the phrase “to detect a gas” may mean detecting, for example, a type and/or a concentration of a gas. Moreover, in the present disclosure, the phrase “to detect a gas” may also mean detecting, for example, the presence of a specific gas, a specific odor or smell of a gas, or the presence and/or the amount of a specific component of a gas. In the present disclosure, the gas detection device may be a device that detects a gas using an electrically driven gas sensor. In the present disclosure, various sensors may be used for the “gas sensor”, which will be described later.

In addition, in the present disclosure, the “gas supply and discharge adapter” may be an adapter to be attached to the above gas sensor. In particular, in the present disclosure, a gas can be supplied to the gas sensor through the “gas supply and discharge adapter”. In the present disclosure, the gas can be discharged from the gas sensor through the “gas supply and discharge adapter”. In other words, in the present disclosure, the gas flows into the “gas supply and discharge adapter”, which enables the gas to be supplied to the gas sensor. In addition, in the present disclosure, the gas flows out of the “gas supply and discharge adapter”, which enables the gas to be discharged from the gas sensor.

A better flow of a gas into and out of the gas sensor leads to an improvement in gas detection of the gas sensor. An object of the present disclosure is to provide a gas supply and discharge adapter and a gas detection device that lead to an improvement in gas detection of a gas sensor. According to one embodiment, the gas supply and discharge adapter and the gas detection device that lead to an improvement in gas detection of the gas sensor can be provided. The gas supply and discharge adapter according to one embodiment will be described with reference to the drawings.

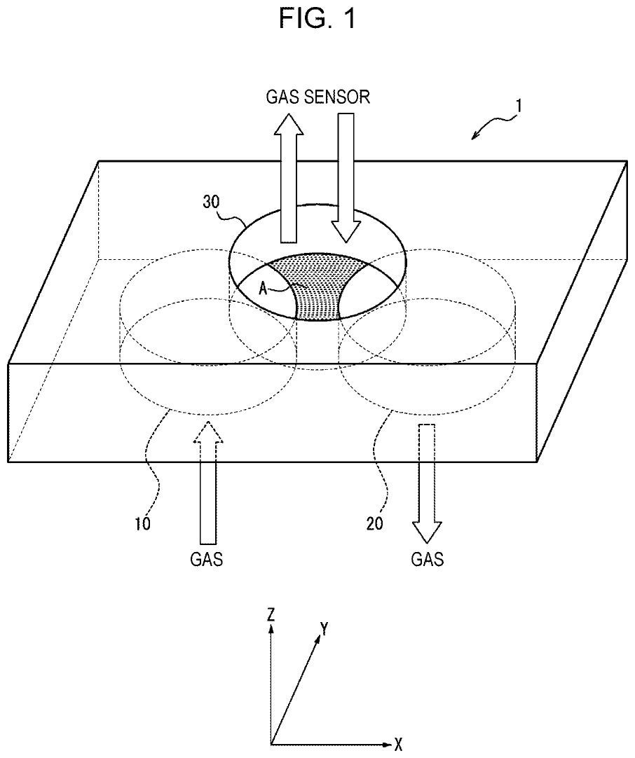

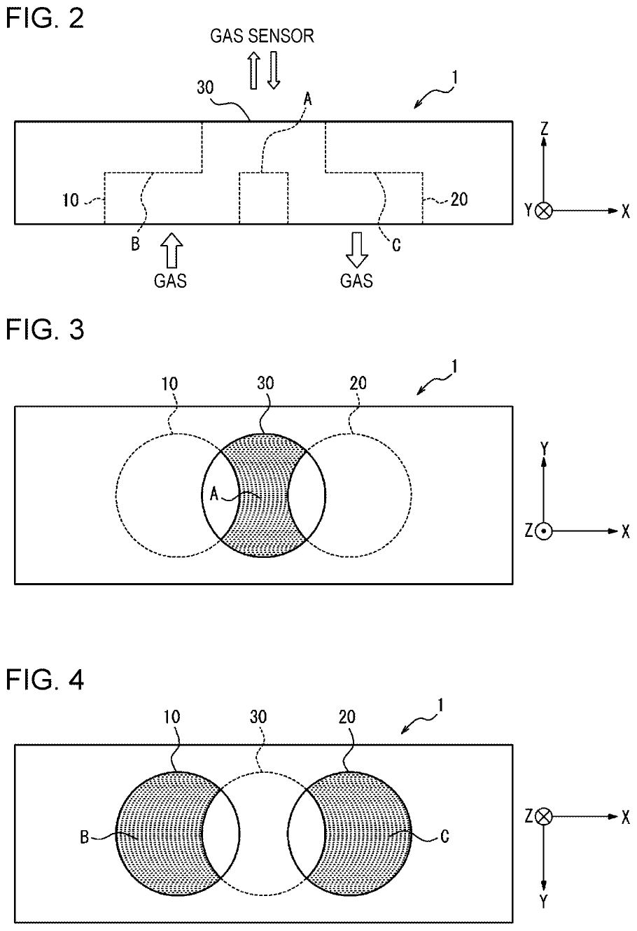

FIG. 1 is a perspective view illustrating the exterior of the gas supply and discharge adapter. FIG. 2 is a front view illustrating the exterior of the gas supply and discharge adapter of FIG. 1. FIG. 3 is a plan view illustrating the exterior of the gas supply and discharge adapter of FIG. 1. FIG. 4 is a bottom view illustrating the exterior of the gas supply and discharge adapter of FIG. 1. FIGS. 1 to 4 illustrate substantially the same gas supply and discharge adapter from different viewpoints.

As illustrated in FIGS. 1 to 4, the exterior of a gas supply and discharge adapter 1 may appear to be, for example, a cuboid. The exterior of the gas supply and discharge adapter 1 is not limited to the cuboid but may assume any shape insofar as the gas supply and discharge adapter 1 can function appropriately. The gas supply and discharge adapter 1 will be described using an example having a substantially rectangular exterior as illustrated in FIGS. 1 to 4.

In the present disclosure, a surface of the gas supply and discharge adapter 1 that faces toward the positive side of the Z-axis in FIG. 1 may be referred to as the “top surface” for convenience's sake. On the other hand, a surface of the gas supply and discharge adapter 1 that faces toward the positive and negative side of the Z-axis in FIG. 1 may be referred to as the “bottom surface” for convenience's sake.

As illustrated in FIGS. 1 to 4, the gas supply and discharge adapter 1 has an inflow port 10, an outflow port 20, and an opening 30. In FIGS. 1 to 4, the inside structure that is not visible from outside the gas supply and discharge adapter 1 is indicated by dotted lines. As illustrated in FIGS. 1 to 4, the inflow port 10 and the outflow port 20 may be formed at the bottom surface of the gas supply and discharge adapter 1. As illustrated in FIGS. 1 to 4, the opening 30 may be formed at the top surface of the gas supply and discharge adapter 1. As illustrated in FIGS. 1 and 2, the inflow port 10 is formed so as to supply a gas. As illustrated in FIGS. 1 and 2, the outflow port 20 is formed so as to discharge the gas. As illustrated in FIGS. 1 and 2, the opening 30 is formed so as to be attached to a gas sensor that detects the gas (which will be described later).

In one embodiment, the gas supply and discharge adapter 1 may be made of various materials. For example, the gas supply and discharge adapter 1 may be made of a resin such as fluororesin, a metal such as aluminum or titan, or a material such as ceramics or glass. The gas supply and discharge adapter 1 defines a channel through which the gas flows, which will be described later. Accordingly, the gas supply and discharge adapter 1 may be made of a material that is durable against deformation or breakage even when a high-temperature or a low-temperature gas flows therethrough.

As described above, the gas sensor is fitted in the opening 30 formed at the top surface of the gas supply and discharge adapter 1. In one embodiment, the gas sensor fitted in the opening 30 may be an arbitrary sensor that can detect the gas supplied from the opening 30. In FIGS. 1 and 2, an illustrations of the gas sensor fitted in the opening 30 is omitted. In FIGS. 1 and 2, flows of the gas through the opening 30 are schematically illustrated with arrows. As illustrated in FIGS. 1 and 2, the gas flows through the opening 30 between the gas supply and discharge adapter 1 and the gas sensor.

As described above, the inflow port 10 is formed so as to supply the gas. In other words, the gas is supplied to the inflow port 10 formed at the bottom surface of the gas supply and discharge adapter 1. A member to be used for supplying the gas may be attached to the inflow port 10. For example, a member, such as a hose, a pipe, a tube, or a duct, may be attached to the inflow port 10 to supply the gas. The member to supply the gas may be any arbitrary member that can supply the gas into the inflow port 10. Accordingly, an illustration of the member is omitted. In FIGS. 1 and 2, a flow of the gas through the inflow port 10 is schematically illustrated with an arrow. As illustrated in FIGS. 1 and 2, the gas is supplied to the gas supply and discharge adapter 1 through the inflow port 10.

As described above, the outflow port 20 is formed so as to discharge the gas. In other words, the gas is discharged from the outflow port 20 formed at the bottom surface of the gas supply and discharge adapter 1. A member to be used for discharging the gas may be attached to the outflow port 20. For example, a member, such as a hose, a pipe, a tube, or a duct, may be attached to the outflow port 20 to discharge the gas. The member to discharge the gas may be any arbitrary member that can discharge the gas from the outflow port 20. Accordingly, an illustration of the member is omitted. In FIGS. 1 and 2, a flow of the gas discharged from the outflow port 20 is schematically illustrated with an arrow. As illustrated in FIGS. 1 and 2, the gas is discharged from the gas supply and discharge adapter 1 through the outflow port 20.

In one embodiment, when the inflow port 10, the outflow port 20, and the opening 30 are formed (bored, for example), the positional relationship and the diameters thereof may be set as follows. As illustrated in FIGS. 3 and 4, the inflow port 10 and the opening 30 may be formed (bored, for example) so as to have such diameters and a positional relationship that the inflow port 10 and the opening 30 at least partially overlap each other as viewed through the gas supply and discharge adapter 1 from above the top surface or from below the bottom surface thereof. Similarly, the outflow port 20 and the opening 30 may be formed (bored, for example) so as to have such diameters and a positional relationship that the outflow port 20 and the opening 30 at least partially overlap each other as viewed through the gas supply and discharge adapter 1 from above the top surface or from below the bottom surface thereof. On the other hand, the inflow port 10 and the outflow port 20 may be formed (bored, for example) so as to have such diameters and a positional relationship that the inflow port 10 and the outflow port 20 do not overlap each other as viewed through the gas supply and discharge adapter 1 from above the top surface or from below the bottom surface thereof. Accordingly, the inflow port 10 and the outflow port 20 may be formed independently of each other.

In addition, as illustrated in FIGS. 1 and 2, the opening 30 may be bored, for example, vertically (in the Z-axis direction) at the top surface of the gas supply and discharge adapter 1. The opening 30 may be bored from the top surface of the gas supply and discharge adapter 1 in the thickness direction thereof (in the Z-axis direction) without piercing the gas supply and discharge adapter 1. For example, as illustrated in FIGS. 1 and 2, the opening 30 is bored approximately halfway into the gas supply and discharge adapter 1 from the top surface in the thickness direction (Z-axis direction) thereof.

As illustrate FIGS. 1 and 2, the inflow port 10 and the outflow port 20 are bored, for example, vertically (in the Z-axis direction) at the bottom surface of the gas supply and discharge adapter 1. The inflow port 10 and the outflow port 20 may be bored from the bottom surface of the gas supply and discharge adapter 1 in the thickness direction thereof (in the Z-axis direction) without piercing the gas supply and discharge adapter 1. For example, as illustrated in FIGS. 1 and 2, the inflow port 10 and the outflow port 20 are bored approximately halfway into the gas supply and discharge adapter 1 from the bottom surface in the thickness direction (Z-axis direction) thereof.

When the inflow port 10 and the opening 30 are formed (bored, for example) as described above, the inflow port 10 and the opening 30 at least partially overlap each other at the connection portion therebetween as illustrated in FIGS. 3 and 4. As illustrated in FIGS. 1 and 2, the inflow port 10 and the opening 30 communicate with each other where the inflow port 10 and the opening 30 at least partially overlap each other. Similarly, when the outflow port 20 and the opening 30 are formed (bored, for example) as described above, the outflow port 20 and the opening 30 at least partially overlap each other at the connection portion therebetween as illustrated in FIGS. 3 and 4. As illustrated in FIGS. 1 and 2, the outflow port 20 and the opening 30 communicate with each other where the outflow port 20 and the opening 30 at least partially overlap each other.

Put another way, as illustrated in FIGS. 1 and 2, when the opening 30 is viewed from above the top surface of the gas supply and discharge adapter 1, the gas supply and discharge adapter 1 is pierced at the overlapping position between the opening 30 and the inflow port 10. In addition, as illustrated in FIGS. 1 and 2, when the opening 30 is viewed from above the top surface of the gas supply and discharge adapter 1, the gas supply and discharge adapter 1 is pierced at the overlapping position between the opening 30 and the outflow port 20. On the other hand, as illustrated in FIGS. 1 and 2, when the opening 30 is viewed from above the top surface of the gas supply and discharge adapter 1, a portion of the gas supply and discharge adapter 1 between the inflow port 10 and the outflow port 20 is not pierced. The unpierced portion between the inflow port 10 and the outflow port 20 is indicated as a portion A in FIGS. 1 and 2.

FIG. 3 is a front view of the gas supply and discharge adapter 1 as viewed from above the top surface thereof (viewing toward the negative side of the Z-axis). As illustrated in FIG. 3, the portion A, which is the unpierced portion between the inflow port 10 and the outflow port 20, is exposed in the opening 30. On the other hand, as illustrated in FIG. 3, the gas supply and discharged adapter 1 is pierced at positions at which the opening 30 overlaps the inflow port 10 and the outflow port 20. Accordingly, the gas can flow into and out of the gas sensor through the opening 30.

In addition, as illustrated in FIGS. 1 and 2, when the inflow port 10 and the outflow port 20 are viewed from below the bottom surface of the gas supply and discharge adapter 1, the gas supply and discharge adapter 1 is pierced at the overlapping positions with the opening 30. Accordingly, the gas can flow into the opening 30 through the inflow port 10. The gas can flow out of the opening 30 through the outflow port 20. On the other hand, as illustrated in FIGS. 1 and 2, when the inflow port 10 and the outflow port 20 are viewed from below the bottom surface of the gas supply and discharge adapter 1, portions of the gas supply and discharge adapter 1 where the inflow port 10 and the outflow port 20 do not overlap the opening 30 are not pierced. The unpierced portion in the inflow port 10, at which the inflow port 10 does not overlap the opening 30, is indicated as a portion B in FIG. 2. In addition, the unpierced portion in the outflow port 20, at which the outflow port 20 does not overlap the opening 30, is indicated as a portion C in FIG. 2.

FIG. 4 is a bottom view of the gas supply and discharge adapter 1 as viewed from below the bottom surface thereof (viewing toward the positive side of the Z-axis). As illustrated in FIG. 4, the portion B, which is the unpierced portion at which the inflow port 10 does not overlap the opening 30, is exposed in the inflow port 10. Similarly, as illustrated in FIG. 4, the portion C, which is the unpierced portion at which the outflow port 20 does not overlap the opening 30, is exposed in the outflow port 20.

In the example illustrated in FIGS. 1 and 2, the connection portion at which the inflow port 10 and the outflow port 20 are connected to the opening 30 is positioned approximately at the middle of the gas supply and discharge adapter 1 in the thickness direction thereof (in the Z-axis direction). In one embodiment, however, the connection portion at which the inflow port 10 and the outflow port 20 are connected to the opening 30 may be formed at an arbitrary position in the thickness direction (Z-axis direction) of the gas supply and discharge adapter 1.

In the above example, it is described that the inflow port 10, the outflow port 20, and the opening 30 are bored in the gas supply and discharge adapter 1. The inflow port 10, the outflow port 20, and the opening 30, however, may be formed in the gas supply and discharge adapter 1 using an arbitrary method. For example, the inflow port 10, the outflow port 20, and the opening 30 are not bored in the gas supply and discharge adapter 1 using a drill but may be formed using a mold.

As described above, the gas supply and discharge adapter 1 may have the inflow port 10, the outflow port 20, and the opening 30. The gas flows into the opening 30 through the inflow port 10. The gas flows out of the opening 30 through the outflow port 20. The gas flows into and out of the gas sensor through the opening 30. The inflow port 10 and the opening 30 may be formed such that the inflow port 10 and the opening 30 at least partially overlap each other at the connection portion therebetween and the gas supply and discharge adapter 1 is pierced through the overlapping portion. The outflow port 20 and the opening 30 may be formed such that the outflow port 20 and the opening 30 at least partially overlap each other at the connection portion therebetween and the gas supply and discharge adapter 1 is also pierced through the overlapping portion.

It may be desired to minimize the supply of a sample gas and/or a purge gas into a gas sensor, such as an odor sensor module or a gas concentration sensor module. It may also be desired to minimize the supply of the sample gas and/or the purge gas, for example, when the size of the module is desired to be reduced. In addition, it is desired to minimize the supply of the sample gas, for example, when the amount of the sample gas is limited. On the other hand, the sensor may be desired to respond quickly even when the supply of the sample gas is small as in the above cases. If the sensor does not respond quickly, the time lag may cause a user, for example, to receive sensing results too late. In addition, in the case where the sensor module that performs waveform analysis is desired to reduce the amount of the gas to be used, the sensor is required to respond to an introduced gas quickly.

In order to reduce the supply of the gas, the following measures may be taken with the gas supply and discharge adapter 1. The gas supply and discharge adapter 1 may be disposed around the portion of the gas sensor that detects the gas, which thereby reduces the volume of a space in which the gas can be diffused compared with the case where the gas sensor is used alone. The gas supply and discharge adapter 1 according to one embodiment is configured to control the velocity of inflow of the gas by adjusting the area of the pierced portion of the opening 30. The gas supply and discharge adapter 1 thereby enables the gas sensor to respond quickly or slowly. In order to retard the gas sensor's response, for example, the opening 30 may be made small, which increases the velocity of inflow of the gas and causes the gas sensor to respond quickly. On the other hand, in order to accelerate the gas sensor's response, for example, the opening 30 may be made large, which decreases the velocity of inflow of the gas and thereby suppresses an abrupt intrusion of the gas into the gas sensor.

In the gas supply and discharge adapter 1, as described above, the inflow port 10 and the outflow port 20 are formed near the opening 30 so as to have partial overlaps with the opening 30 that is connected to the gas sensor. In other words, the inflow port 10 and the outflow port 20 are formed so as to overlap the opening 30 at least partially as viewed in plan through the gas supply and discharge adapter 1 from above the top surface or from below the bottom surface thereof. When the inflow port 10 and the opening 30 are viewed in plan through the gas supply and discharge adapter 1, at least a portion of the inflow port 10 may overlap the opening 30. In particular, when the inflow port 10 and the opening 30 are viewed in plan through the gas supply and discharge adapter 1, a portion of the inflow port 10 may overlap the opening 30. In addition, when the outflow port 20 and the opening 30 are viewed in plan through the gas supply and discharge adapter 1, at least a portion of the outflow port 20 may overlap the opening 30. In particular, when the outflow port 20 and the opening 30 are viewed in plan through the gas supply and discharge adapter 1, a portion of the outflow port 20 may overlap the opening 30. In addition, the inflow port 10 and the outflow port 20 may be disposed closely, for example, so as to be adjacent to each other. This leads to a reduction in the size of the gas supply and discharge adapter 1, which further leads to an reduction in the supply of the sample gas.

In addition, in the gas supply and discharge adapter 1, the size (area) of the partially overlapping portion, which is the pierced portion, between the inflow port 10 and the opening 30 may be changed appropriately. Moreover, in the example illustrated in FIGS. 1 and 2, reducing the size of the opening 30 can increase the velocity of the gas flowing into the gas sensor through the opening 30. With this configuration, the gas sensor's response can be accelerated due to the velocity increase of the sample gas flowing into the gas sensor through the opening 30.

On the other hand, in the example illustrated in FIGS. 1 and 2, increasing the size of the opening 30 can decrease the velocity of the gas flowing into the gas sensor through the opening 30. With this configuration, the gas sensor's response can be retarded due to the velocity decrease of the sample gas flowing into the gas sensor through the opening 30. This can suppress an overreaction of the gas sensor, for example, in an early stage of the gas sensor's response.

As described above, the gas supply and discharge adapter 1 enables the gas to flow favorably into and out of the gas sensor. Thus, a gas supply and discharge adapter that leads to an improvement in gas detection of the gas sensor can be provided in accordance with the gas supply and discharge adapter 1.

Next, gas supply and discharge adapters according to other embodiments will be described.

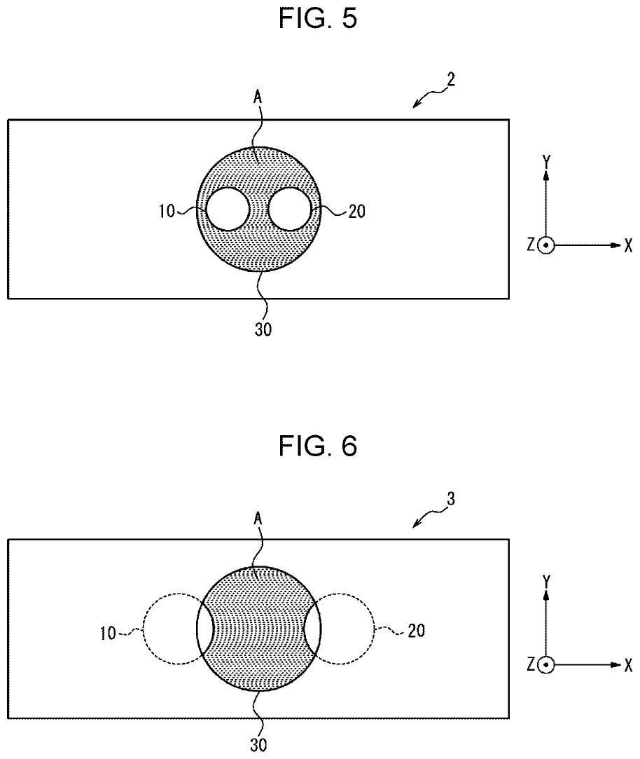

FIG. 5 is a front view of a gas supply and discharge adapter according to one embodiment as viewed from above the top surface thereof (viewing toward the negative side of the Z-axis) as is the case for FIG. 3.

As illustrated in FIG. 5, the inflow port 10 and the opening 30 may be formed (bored, for example) so as to have such diameters and a positional relationship that the inflow port 10 entirely overlaps the opening 30 as viewed through a gas supply and discharge adapter 2 from above the top surface thereof. As illustrated in FIG. 5, the outflow port 20 and the opening 30 may be also formed (bored, for example) so as to have such diameters and a positional relationship that the outflow port 20 entirely overlaps the opening 30 as viewed through the gas supply and discharge adapter 2 from above the top surface thereof. As illustrated in FIG. 5, the inflow port 10 and the outflow port 20 may be formed adjacent to each other in the gas supply and discharge adapter 2.

With the configuration illustrated in FIG. 5, the gas flowing through the inflow port 10 and the outflow port 20 does not tend to be obstructed. Accordingly, the gas supply and discharge adapter 2 having the configuration illustrated in FIG. 5 is especially beneficial in the case where the response of the sensor needs to be expedited by reducing the size of the opening 30.

FIG. 6 is a front view of a gas supply and discharge adapter according to one embodiment as viewed from above the top surface thereof (viewing toward the negative side of the Z-axis) as are the cases for FIGS. 3 and 5. In FIG. 6, the inside structure that is not visible from outside is indicated by dotted lines.

Accordingly, when the inflow port 10 and the opening 30 are viewed in plan through the gas supply and discharge adapter, at least a portion of the inflow port 10, or especially the entirety of the inflow port 10, may overlap the opening 30. In addition, when the outflow port 20 and the opening 30 are viewed in plan through the gas supply and discharge adapter, at least a portion of the outflow port 20, or especially the entirety of the outflow port 20, may overlap the opening 30.

As illustrated in FIG. 6, the inflow port 10 and the opening 30 may be formed (bored, for example) so as to have such diameters and a positional relationship that the inflow port 10 and the opening 30 overlap each other only slightly as viewed through a gas supply and discharge adapter 3 from above the top surface thereof. In addition, the outflow port 20 and the opening 30 may be formed (bored, for example) so as to have such diameters and a positional relationship that the outflow port 20 and the opening 30 overlap each other only slightly as viewed through the gas supply and discharge adapter 3 from above the top surface thereof.

With the configuration illustrated in FIG. 6, the gas flowing through the inflow port 10 and the outflow port 20 tends to be obstructed. Accordingly, the gas supply and discharge adapter 3 having the configuration illustrated in FIG. 6 is especially beneficial in the case where the overresponse of the sensor needs to be controlled by increasing the size of the opening 30.

In the gas supply and discharge adapter 2 of FIG. 5, the sizes of the inflow port 10 and the outflow port 20 are set to be smaller than the size of the opening 30. In the gas supply and discharge adapter 3 of FIG. 6, the sizes of the inflow port 10 and the outflow port 20 are also set to be smaller than the size of the opening 30. On the other hand, in the gas supply and discharge adapter 1 of FIG. 1, the sizes of the inflow port 10 and the outflow port 20 are set to be substantially equal to the size of the opening 30. Accordingly, in one embodiment, the sizes of the inflow port 10 and the outflow port 20 may be set to be equal to, or smaller than, the size of the opening 30.

In one embodiment, the inflow port 10 and the outflow port 20 may be formed adjacent to each other. In one embodiment, the size of at least one of the inflow port 10 and the outflow port 20 may be equal to or smaller than the size of the opening 30.

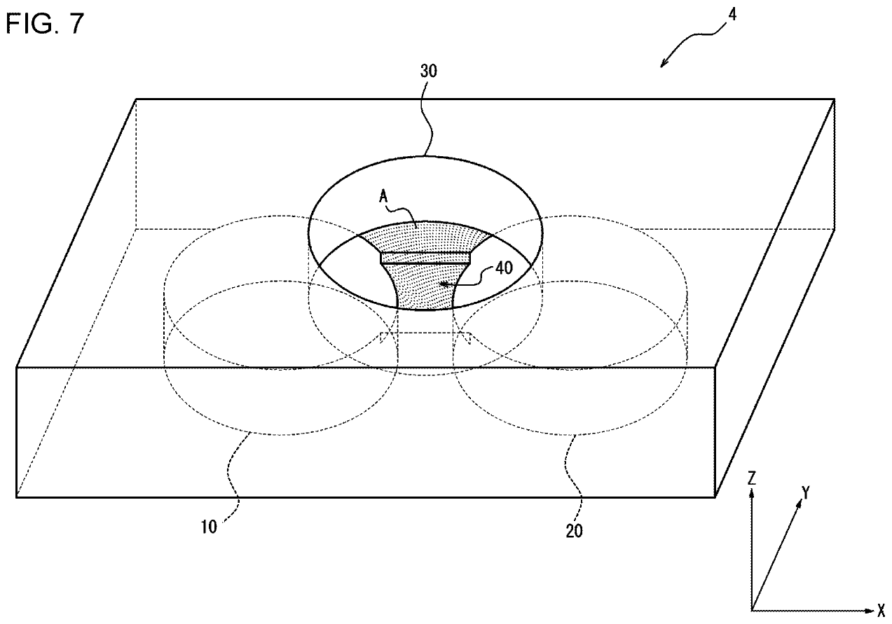

FIG. 7 is a front view of a gas supply and discharge adapter according to one embodiment as viewed from above the top surface thereof (viewing toward the negative side of the Z-axis) as is the case for FIG. 1.

As illustrated in FIG. 7, a gas supply and discharge adapter 4 may have a groove 40 formed at the unpierced portion A in the opening 30 between the inflow port 10 and the outflow port 20. The groove 40 may have an appropriate size so that the gas can flow appropriately from the inflow port 10 toward the outflow port 20. For example, the groove 40 may have a depth of 0.2 mm in the Z-axis direction. The width in the Y-axis direction of the groove 40 may be substantially equal to the radius of the inflow port 10 and/or the outflow port 20. The depth of the groove 40 in the Z-axis direction may be set to be greater or smaller than 0.2 mm depending on the necessity. The width of the groove 40 in the Y-axis direction may be set to be larger or smaller than the radius of the inflow port 10 and/or the outflow port 20. The groove 40, however, may be sized so as to enable the gas to flow appropriately from the inflow port 10 toward the outflow port 20. The shape of the groove 40 is not limited to that illustrated in FIG. 7. The groove 40 may be shaped arbitrarily insofar as the gas flows appropriately from the inflow port 10 toward the outflow port 20.

As described above, the gas supply and discharge adapter 4 may have the groove 40 formed from the inflow port 10 to the outflow port 20 at the connection portion at which the inflow port 10 and the outflow port 20 are connected to the opening 30. In this case, the groove 40 may be formed so as to ensure gas communication therebetween.

For example, when the gas sensor is fitted in the opening 30 of the gas supply and discharge adapter, a filter, such as a filter for removing an unneeded gas, a dust trap filter, or a wire net, may be installed between the gas sensor and the opening 30. In the case of the above filter being installed so as to be exposed directly to the inflow port 10 and the outflow port 20, the filter may cause pressure drop. In such a case, the groove 40 formed as described above may mitigate the pressure drop. In addition, the above groove 40 may also facilitate the flow of the gas supplied from the opening 30 to the gas sensor and/or discharged from the gas sensor to the opening 30.

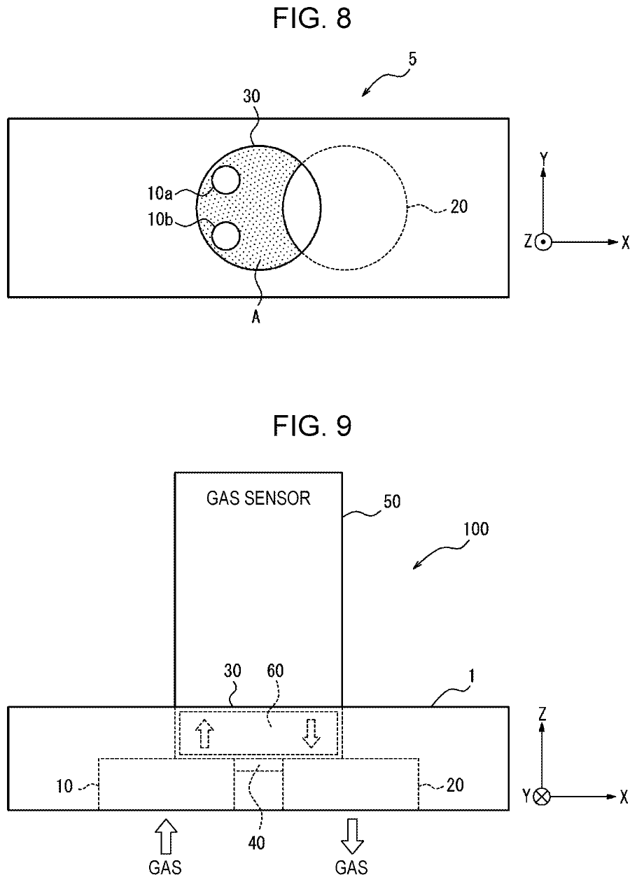

FIG. 8 is a front view of the gas supply and discharge adapter according to one embodiment as viewed from above the top surface thereof (viewing toward the negative side of the Z-axis) as are the cases for FIGS. 3 and 5.

A gas supply and discharge adapter 5 illustrated in FIG. 8 may have two inflow ports, in other words, an inflow port 10a and an inflow port 10b. In addition, as illustrated in FIG. 8, the inflow port 10a, the inflow port 10b, and the opening 30 may be formed (bored, for example) so as to have such diameters and a positional relationship that the inflow port 10a and the inflow port 10b entirely overlap the opening 30 as viewed through the gas supply and discharge adapter 5 from above the top surface thereof. As illustrated in FIG. 8, the gas supply and discharge adapter 5 may have one outflow port 20. In addition, as illustrated in FIG. 8, the outflow port 20 and the opening 30 may be formed (bored, for example) so as to have such diameters and a positional relationship that the outflow port 20 and the opening 30 partially overlap each other as viewed through the gas supply and discharge adapter 5 from above the top surface thereof.

As described above, in one embodiment, a plurality of inflow ports 10 may be formed. In one embodiment, three or more inflow ports 10 may be formed. When the inflow ports 10 and the opening 30 are viewed in plan through the gas supply and discharge adapter, at least a portion of each inflow port 10, or especially the entirety of each inflow port 10, may overlap the opening 30. In this case, only one outflow port 20 may be formed. When the outflow port 20 and the opening 30 are viewed in plan through the gas supply and discharge adapter, at least a portion of the outflow port 20 may overlap the opening 30. In particular, when the outflow port 20 and the opening 30 are viewed in plan through the gas supply and discharge adapter 1, a portion of the outflow port 20 may overlap the opening 30.

Next, a gas detection device according to one embodiment will be described.

As illustrated in FIG. 9, a gas detection device 100 includes the gas supply and discharge adapter 1 and a gas sensor 50. The gas detection device 100 according to one embodiment may also include a filter 60. The gas supply and discharge adapter 1 may be the same one as described in relation to FIGS. 1 to 4. In one embodiment, any one of the gas supply and discharge adapters 2 to 5 described in relation to FIGS. 5 to 8 may be adopted here in place of the gas supply and discharge adapter 1. Since the gas supply and discharge adapter 1 and others have already been described, detailed descriptions will be omitted.

The gas sensor 50 may be an arbitrary gas sensor to be fitted in the gas supply and discharge adapter 1 or the like. More specifically, the gas sensor 50 is fitted in the opening 30 of the gas supply and discharge adapter 1 or the like. In other words, the gas detection device 100 may be formed by attaching the gas sensor 50 to the above-described gas supply and discharge adapter 1 or the like.

As illustrated in FIG. 9, the gas sensor 50 may be configured to be appropriately fitted in the opening 30. As illustrated in FIG. 9, a sensing portion (or a filter 60) of the gas sensor 50 is desirably disposed, for example, so as to be in contact with (or in proximity with) the portion A as illustrated in FIG. 7 in an aim to reduce the supply of the gas. For example, a filter or a mesh or the like is present at an end portion of the gas sensor 50 that is in contact with the portion A. In this configuration, the filter 60 or a material (for example, a wire mesh or the like) for protecting the sensing portion of the gas sensor 50 causes pressure drop. The provision of the groove 40, however, can reduce the likelihood of the pressure drop.

As described above, the gas detection device 100 has the inflow port 10, the outflow port 20, and the opening 30, and also includes the gas sensor 50. The gas sensor 50 detects the gas supplied to the opening 30. The gas flows into the opening 30 through the inflow port 10. The gas flows out of the opening 30 through the outflow port 20. In the gas detection device 100, the inflow port 10, the outflow port 20, and the opening 30 may be formed in the same manner as those described in the gas supply and discharge adapter 1 or the like.

The present disclosure has been described with reference to the drawings and examples. It should be noted that one skilled in the art can easily make various modifications and alterations on the basis of the present disclosure. Accordingly, such modifications and alterations are to be included in the scope of the present disclosure. For example, functions contained in functional parts may be reallocated insofar as such reallocation does not lead to a logical inconsistency. Multiple functional parts or the like may be combined into one or may be further divided. The above-described embodiments of the present disclosure are not meant to be implemented precisely in accordance with the description but can be implemented while features contained in the embodiments are combined with one another or some features are omitted in an appropriate manner.

### REFERENCE SIGNS LIST

1, 2, 3, 4, 5 gas supply and discharge adapter

10 inflow port

20 outflow port

30 opening

40 groove

50 gas sensor

60 filter

100 gas detection device

1. A gas supply and discharge adapter, comprising:
an opening through which a gas flows into and out of a gas sensor;
an inflow port through which the gas flows into the opening; and
an outflow port through which the gas flows out of the opening, wherein
the inflow port and the outflow port are positioned independently of each other, and
when the inflow port and the opening are viewed in plan through the gas supply and discharge adapter, at least a portion of the inflow port overlaps the opening.

2. The gas supply and discharge adapter according to claim 1, wherein
when the inflow port and the opening are viewed in plan through the gas supply and discharge adapter, a portion of the inflow port overlaps the opening.

3. The gas supply and discharge adapter according to claim 1, wherein
when the outflow port and the opening are viewed in plan through the gas supply and discharge adapter, at least a portion of the outflow port overlaps the opening.

4. The gas supply and discharge adapter according to claim 3, wherein
when the outflow port and the opening are viewed in plan through the gas supply and discharge adapter, a portion of the outflow port overlaps the opening.

5. The gas supply and discharge adapter according to claim 1, wherein
a size of at least one of the inflow port and the outflow port is equal to or smaller than a size of the opening.

6. The gas supply and discharge adapter according to claim 1, further comprising:
a groove formed so as to extend from the inflow port to the outflow port at a connection portion at which the inflow port and the outflow port are connected to the opening.

7. The gas supply and discharge adapter according to claim 6, wherein
the groove is positioned so as to ensure gas communication.

8. The gas supply and discharge adapter according to claim 1, wherein
the total number of the inflow ports is two or more.

9. The gas supply and discharge adapter according to claim 1, wherein
the total number of the outflow ports is one.

10. A gas detection device, comprising:
a gas sensor that detects a gas supplied to an opening;
an inflow port through which the gas flows into the opening; and
an outflow port through which the gas flows out of the opening; wherein
the inflow port and the outflow port are positioned independently of each other, and
when the inflow port and the opening are viewed in plan through the gas supply and discharge adapter, at least a portion of the inflow port overlaps the opening.

11. The gas detection device according to claim 10, wherein
when the inflow port and the opening are viewed in plan through the gas supply and discharge adapter, a portion of the inflow port overlaps the opening.

12. The gas detection device according to claim 10, wherein
when the outflow port and the opening are viewed in plan through the gas supply and discharge adapter, at least a portion of the outflow port overlaps the opening.

13. The gas detection device according to claim 12, wherein
when the outflow port and the opening are viewed in plan through the gas supply and discharge adapter, a portion of the outflow port overlaps the opening.

14. The gas detection device according to claim 10, wherein
a filter to remove an unneeded gas is disposed between the gas sensor and the opening.

15. The gas detection device according to claim 10, further comprising:
a groove formed so as to extend from the inflow port to the outflow port at a connection portion at which the inflow port and the outflow port are connected to the opening.
